(19) United States
(12) Patent Application Publication
(10) Pub. No.: US 2023/0141144 A1
(43) Pub. Date: May 11, 2023

(54) SOLID CLEANSING COMPOSITION COMPRISING AN ACYL ALKYL ISETHIONATE

(71) Applicant: Innospec Active Chemicals LLC, High Point, NC (US)

(72) Inventors: Phillip Loraine Cotrell, Salisbury, NC (US); Kimi Aileen Yamada, Waxhaw, NC (US)

(21) Appl. No.: 17/800,111

(22) PCT Filed: Mar. 11, 2021
(86) PCT No.: PCT/GB2021/050601

§ 371 Date: Aug. 16, 2022

Related U.S. Application Data
(60) Provisional application No. 62/988,471, filed on Mar. 12, 2020.

(51) Int. Cl. A61K 8/46; A61K 8/44; A61K 8/365; A61K 8/19; A61K 8/92; A61Q 5/02; A61Q 19/10; A61K 8/04; A61K 8/02

(52) U.S. Cl. CPC A61K 8/466; A61K 8/19; A61K 8/022; A61K 8/046; A61K 8/365; A61K 8/447; A61K 8/463; A61K 8/922; A61Q 5/02; A61Q 19/10; A61K 2800/87; A61K 2800/222; A61K 2800/596

(57) ABSTRACT

A solid cleansing composition comprising:
(i) at least one acyl alkyl isethionate surfactant of the formula (I):

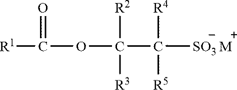

wherein R1 represents an optionally substituted C4-C36 hydrocarbyl group;
each of R2, R3, R4 and R5 independently represents hydrogen or a C1-C4 alkyl group and
wherein at least one of R2, R3, R4 and R5 is not hydrogen; and M+ represents a cation;
(ii) a salt of carbonic acid; and
(iii) an organic acid.

### FIELD OF THE INVENTION

The present invention relates to solid cleansing compositions. The present invention also relates to a cleansing product comprising a solid cleansing composition, a method of cleansing skin and/or hair, the use of a solid cleansing composition for cleansing skin and/or hair, and a method of manufacturing a cleansing product.

### BACKGROUND OF THE INVENTION

Many commonly available cleansing products (such as shampoos, body washes and the like) are in the form of viscous liquid compositions. Such compositions are easy to dispense. However, a user will often pour a much larger volume than they intended of the composition onto their hand prior to application, for example to the hair or body, and thus significant quantities are wasted. Liquid compositions are heavy and often contain large volumes of water. It is expensive and environmentally unfriendly to transport large volumes of liquid, and the use of large quantities of water during manufacturing is detrimental to the environment.

Liquid cleansing compositions are usually packaged in plastic bottles. Plastic bottles are durable, flexible, and easy to manufacture in a variety of shapes. However, most plastic bottles are derived from petrochemicals and not from a sustainable source. Plastic bottles are typically not biodegradable and plastic bottles which are discarded will typically persist in the environment for a long period of time. Plastic bottles may be recycled, but this is energy- and labour-intensive and currently only a small proportion of plastic bottles are recycled.

Solid cleansing compositions offer significant advantages over liquid compositions. They are more compact, require less packaging (especially less plastic packaging) and are easy to transport. In addition, a user typically only uses the amount of the composition needed and thus there is a reduction in waste. Solid cleansing compositions are not stored and used in bottles, which, along with the overall reduction in packaging, makes them easier to use and apply. This provides advantages especially for use by older people and for application to animals. It is easy to provide solid compositions as single use products.

Known solid cleansing compositions include soap bars and sulfate surfactant-based solid shampoo compositions. However, the surfactants in such compositions are quite harsh and may cause irritancy to the skin of a user.

Low-irritancy solid cleansing compositions typically have inadequate foam-forming properties when brought into contact with water and/or the body of a user. It may take a long time and considerable effort for a user to achieve the desired level of foam, and the user may not be able to fully apply the cleansing composition to the desired part of the body.

Therefore, there remains a need for solid cleansing compositions which are low-irritancy, yet quick and easy to use, and which form desirable foams in use.

It is an aim of the present invention to provide an improved solid cleansing composition.

### SUMMARY OF THE INVENTION

According to a first aspect of the present invention, there is provided a solid cleansing composition comprising:
(i) at least one acyl alkyl isethionate surfactant of the formula (I):

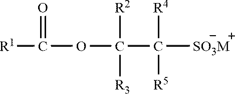

wherein R1 represents an optionally substituted C4-C36 hydrocarbyl group; each of R2, R3, R4 and R5 independently represents hydrogen or a C1-C4 alkyl group and wherein at least one of R2, R3, R4 and R5 is not hydrogen; and M+ represents a cation;
(ii) a salt of carbonic acid; and
(iii) an organic acid.

According to a second aspect of the present invention, there is provided a cleansing product comprising a solid cleansing composition according to the first aspect and packaging.

According to a third aspect of the present invention, there is provided a method of cleansing skin and/or hair comprising the steps of:
(a) contacting the composition according to the first aspect with water to form a cleansing foam; and
(b) contacting the cleansing foam with the skin and/or hair.

According to a fourth aspect of the present invention, there is provided a use of a composition according to the first aspect for cleansing skin and/or hair.

According to a fifth aspect of the present invention, there is provided a method of manufacturing a solid cleansing product, the method comprising the steps of:
(a) forming an admixture comprising:
   (i) at least one acyl alkyl isethionate surfactant of the formula (I): 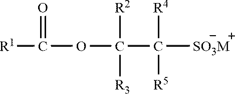
   wherein R1 represents an optionally substituted C4-C36 hydrocarbyl group; each of R2, R3, R4 and R5 independently represents a hydrogen atom or a C1-C4 alkyl group and wherein at least one of R2, R3, R4 and R5 is not hydrogen; and M+ represents a cation;
   (ii) a salt of carbonic acid; and
   (iii) an organic acid; and
(b) enclosing the admixture in packaging.

### DETAILED DESCRIPTION OF THE INVENTION

Unless otherwise stated, the following terms used in the specification and claims have the meanings set out below.

The term “fatty acid” is used herein in its ordinary sense, which is well-known to those skilled in the art. Specifically, it refers to a nonesterified fatty acid.

The term “organic acid” is used herein in its ordinary sense, which is well known to those skilled in the art. Specifically, it refers to an organic compound with acidic properties.

As used herein, the term “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include hydrocarbon groups, i.e. aliphatic (which may be saturated or unsaturated, linear or branched, for example alkyl or alkenyl), alicyclic (for example cycloalkyl, cycloalkenyl) and aromatic (for example phenyl) groups.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C4-C36 alkyl” includes C10-C36, C4-C6 alkyl, propyl, isopropyl and t-butyl.

The term “alkenyl” includes both straight and branched chain alkenyl groups. References to individual alkenyl groups such as “propenyl” are specific for the straight chain version only and references to individual branched chain alkenyl groups such as “isopropenyl” are specific for the branched chain version only. For example, “C4-C36 alkenyl” includes C10-C36 alkenyl, C4-C6 alkenyl, propenyl and isopropenyl.

The term “alkoxy” includes both straight and branched chain alkoxy groups. References to individual alkoxy groups such as “propoxy” are specific for the straight chain version only and references to individual branched chain alkyl groups such as “isopropoxy” are specific for the branched chain version only. For example, “C1-C4 alkoxy” includes C1-C2 alkoxy, propoxy, isopropoxy and t-butoxy.

The term “aryl” means a cyclic or polycyclic aromatic ring having from 5 to 12 carbon atoms. Examples of aryl groups include, but are not limited to, phenyl, biphenyl and naphthyl. In a particular embodiment, an aryl group may be phenyl.

The term “C8-C22 alkyl-C6-C12 aryl” means a C6-C12 aryl group covalently attached to a C8-C22 alkyl group, both of which are defined herein.

The term “optionally substituted” with reference to a particular group, such as a hydrocarbyl group, alkyl group, alkenyl group, alkoxy group, or aryl group means that said group may be substituted or unsubstituted. Suitable substituents may include non-hydrocarbon groups provided that they do not alter the predominantly hydrocarbon nature of the group. Examples of suitable substituents include C1-4 alkoxy, cyano, hydroxy, oxo, halo (especially fluoro and chloro), trifluoromethyl and trifluoromethoxy.

Unless stated to be optionally substituted, the hydrocarbyl groups, alkyl groups, alkenyl groups, alkoxy groups, and aryl groups herein are unsubstituted.

References to a solid composition herein refer to compositions which are in the solid state under normal atmospheric conditions (i.e. at a pressure of 1 atmosphere and 298 K).

References to “soap” herein refer to compounds commonly known as soap, for example the alkali metal, alkaline earth metal, ammonium, ammonium hydroxide and alkanol ammonium salts of aliphatic alkane or alkene monocarboxylic acids.

Throughout this specification, the term “comprising” or “comprises” means including the component(s) specified but not to the exclusion of the presence of other components. The term “consisting essentially of” or “consists essentially of” means including the components specified but excluding other components except for components added for a purpose other than achieving the technical effect of the invention. The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

For the avoidance of doubt, where amounts of components in a composition are described in wt%, this means the weight percentage of the specified component in relation to the whole composition referred to. For example, “wherein the solid cleansing composition comprises from 5 to 50 wt% of at least one acyl alkyl isethionate surfactant of the formula (I)” means that 5 to 50 wt% of the solid cleansing composition is provided by at least one acyl alkyl isethionate of the formula (I).

In this specification, unless otherwise indicated any amounts referred to relate to the amount of active component present in the composition. The skilled person will appreciate that commercial sources of some of the components referred to herein may include impurities, side-products and/or residual starting material. However, the amounts specified refer only to the active material and do not include any impurity, side-product, starting material or diluent that may be present.

The optional features set out herein may be used either individually or in combination with each other where appropriate and particularly in the combinations as set out in the accompanying claims. The optional features for each exemplary embodiment of the invention, as set out herein are also applicable to any other aspects or exemplary embodiments of the invention, where appropriate. In other words, the skilled person reading this specification should consider the optional features for each aspect or embodiment of the invention as interchangeable and combinable between different aspects of the invention.

### Solid Cleansing Composition

According to a first aspect of the present invention, there is provided a solid cleansing composition comprising:
(i) at least one acyl alkyl isethionate surfactant of the formula (I):

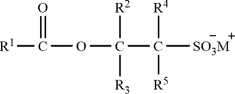

wherein R1 represents an optionally substituted C4-C36 hydrocarbyl group; each of R2, R3, R4 and R5 independently represents hydrogen or a C1-C4 alkyl group and wherein at least one of R2, R3, R4 and R5 is not hydrogen; and M+ represents a cation;
(ii) a salt of carbonic acid; and
(iii) an organic acid.

The solid cleansing composition of the first aspect may be in a compacted, crystalline or powdered form. Suitable compacted forms include bars, blocks, pucks, and sticks, and may be formed by compressing an admixture of the components of the composition. Suitably the composition is in a crystalline or powdered form, for example a powder. The solid cleansing composition is advantageously lighter and more compact than a liquid cleansing composition comprising the same amount of surfactant. The solid cleansing composition advantageously requires less packaging than liquid cleansing compositions, and may even require no packaging at all.

The solid cleansing composition of the first aspect provides desirable foaming ability in use and/or may dissolve in suitable amounts of water without leaving any residue/undissolved solids present.

Suitably, the solid cleansing composition of the first aspect is for cleaning the skin and/or hair of a human or animal (such as a pet). Suitably, the solid cleansing composition of the first aspect is a personal cleansing composition, for example which is suitable for cleansing skin and/or hair. In some embodiments the solid cleansing composition of the first aspect is a shampoo, a body wash, a hand cleanser, a facial cleanser, a skin cleanser, a shaving composition, or a general personal cleanser.

Suitably, the solid cleansing composition of the first aspect is effervescent. By this we mean that the composition forms a foam upon contact with water. It is believed that the salt of carbonic acid and the organic acid present in the composition react together in the presence of water to produce carbon dioxide, which is encapsulated by a mixture of the water and the surfactant of the formula (I) to form a foam.

The solid cleansing composition of the first aspect is believed to provide a solid cleansing composition which rapidly forms a stable, creamy foam upon contact with water through effervescence. Advantageously the foam is produced without the need for force to be applied to the composition.

Suitably, the cleansing composition of the first aspect is substantially anhydrous. By this we mean that the composition does not intentionally comprise water, i.e. the cleansing composition of the first aspect comprises less than 5 wt%, such as less than 2 wt%, for example less than 1 wt%, of water.

Suitably, the solid cleansing composition of the first aspect comprises from 5 to 50 wt% of at least one acyl alkyl isethionate surfactant of the formula (I), such as from 10 to 45 wt% of at least one acyl alkyl isethionate surfactant of the formula (I), particularly from 10 to 35 wt% of at least one acyl alkyl isethionate surfactant of the formula (I).

In the formula (I), R1 represents an optionally substituted C4-C36 hydrocarbyl group, R2, R3, R4 and R5 each independently represents hydrogen or a substituted or unsubstituted C1-C4 alkyl group, provided that at least one of R2, R3, R4 and R5 is not hydrogen, and M+ represents a cation.

Suitably, R1 represents an optionally substituted C4-C36 alkyl, C4-C36 alkenyl, C6-C12 aryl or C8-C22 alkyl-C6-C12 aryl group. More suitably, R1 represents an optionally substituted C4-C36 alkyl or C4-C36 alkenyl group, especially an optionally substituted C4-C36 alkyl group. Most suitably, R1 represents a C4-C36 alkyl or C4-C36 alkenyl group, especially a C4-C36 alkyl group.

Suitably, R1 represents an optionally substituted C4-C36 alkyl or C4-C36 alkenyl group, such as an optionally substituted C8-C18 alkyl or C8-C18 alkenyl group.

Suitably, R1 represents a C4-C36 alkyl or C4-C36 alkenyl group, such as a C8-C18 alkyl or C8-C18 alkenyl group.

Suitably, R1 represents an optionally substituted C5-C30 alkyl group, such as an optionally substituted C7-C24 alkyl group, for example an optionally substituted C7-C21 alkyl group, preferably an optionally substituted C7-C17 alkyl group.

Suitably, R1 represents a C5-C30 alkyl group, such as a C7-C24 alkyl group, for example a C7-C21 alkyl group, preferably a C7-C17 alkyl group.

R1 is suitably the residue of a fatty acid. Fatty acids obtained from natural oils often include mixtures of fatty acids. For example, the fatty acid obtained from coconut oil contains a mixture of fatty acids including C12 lauric acid, C14 myristic acid, C16 palmitic acid, C8 caprylic acid, C10 capric acid and C18 stearic and oleic acid.

R1 may include the residue of one or more naturally occurring fatty acids and/or of one or more synthetic fatty acids. For example, R1 may consist essentially of the residue of a single fatty acid.

Examples of carboxylic acids from which R1 may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R1 comprises the residue of coco acid, the residue of mixed fatty acids derived from coconut oil or the residue of mixed fatty acids derived from palm kernel oil. More suitably, R1 predominantly comprises the residue of a saturated fatty acid having 12 carbon atoms.

The acyl alkyl isethionate surfactant of the formula (I) may be prepared by any of the methods disclosed in the prior art, for example see the methods described in WO94/09763 and WO2005/075623.

In some embodiments only a single acyl alkyl isethionate surfactant of the formula (I) may be present in the solid cleansing composition of the first aspect. In some embodiments a mixture of two or more acyl alkyl isethionate surfactants of the formula (I) may be present. In such embodiments the above amounts refer to the total amounts of all acyl alkyl isethionate surfactants of the formula (I) present in the composition.

When any of R2, R3, R4 and R5 represents an optionally substituted C1-C4 alkyl group, the alkyl group is suitably n-propyl, ethyl or methyl, such as ethyl or methyl, most preferably methyl.

Preferably one of the groups R2, R3, R4 and R5 represents an optionally substituted C1-C4 alkyl group and the remaining groups represent hydrogen. For example, R2 may represent an optionally substituted C1-C4 alkyl group and R3, R4 and R5 may all represent hydrogen. For example, R4 may represent an optionally substituted C1-C4 alkyl group and R2, R3 and R5 may all represent hydrogen.

Preferably, R2 represents a C1-C4 alkyl group and R3, R4 and R5 all represent hydrogen. Preferably, R4 represents a C1-C4 alkyl group and R2, R3 and R5 all represent hydrogen.

Most preferably, R2 represents a methyl group and R3, R4 and R5 all represent hydrogen. Most preferably, R4 represents a methyl group and R2, R3 and R5 all represent hydrogen. For example, the acyl alkyl isethionate surfactant of the formula (I) may be selected from one or more of sodium lauroyl methyl isethionate, sodium cocoyl methyl isethionate and sodium oleoyl methyl isethionate. Sodium lauroyl methyl isethionate is especially preferred.

Suitably, M+ represents a metal cation or an optionally substituted ammonium cation,preferably a metal cation. By “optionally substituted ammonium cation”, we mean to refer to an ammonium cation wherein the nitrogen atom may be substituted with from 1 to 4 optionally substituted hydrocarbyl groups. Suitable ammonium cations include NH4+ and the ammonium cation of triethanolamine. Suitable metal cations include alkali metal cations, for example sodium, lithium and potassium cations, and alkaline earth metal cations, for example calcium and magnesium cations. Suitably, M+ represents an alkali metal cation or an optionally substituted ammonium cation. Preferably, M+ represents a zinc, potassium or sodium cation. Most preferably, M+ represents a sodium cation.

The skilled person will appreciate that when M+ is a divalent metal cation two moles of anion will be present for each mole of cation.

The acyl alkyl isethionate surfactant of formula (I) may comprise the reaction product of sodium methyl isethionate and a fatty acid, that is a compound of formula R1COOCHR2CHR4SO3-M+ in which one of R2 and R4 is methyl and the other is hydrogen. Mixtures of these isomers may be present.

The solid cleansing composition of the present invention may include a mixture of more than one acyl alkyl isethionate surfactant of formula (I). For example, an isomeric mixture of acyl alkyl isethionate surfactants of formula (I) may be present. Such a mixture may include, for example an acyl alkyl isethionate surfactant in which R2 represents a C1-C4 alkyl group (suitably methyl) and R3, R4 and R5 are all hydrogen and an acyl alkyl isethionate surfactant in which R4 represents a C1-C4 alkyl group (suitably methyl) and R2, R3 and R5 are all hydrogen.

In particular, the solid cleansing composition of the present invention may comprise a mixture of isomers, that is a compound of formula R1COOCH2CHR4SO3-M+ in which R4 represents a C1-C4 alkyl group (preferably methyl) and a compound of formula R1COOCHR2CH2SO3-M+ in which R2 represents a C1-C4 alkyl group (preferably methyl).

Suitably such mixtures comprise approximately 90% of compounds in which R2 is methyl and R4 is hydrogen and approximately 10% of compounds in which R2 is hydrogen and R4 is methyl.

Suitably, the solid cleansing composition of the first aspect comprises from 30 to 60 wt% of the salt of carbonic acid, more suitably from 35 to 57 wt% of the salt of carbonic acid, such as from 40 to 55 wt% of the salt of carbonic acid.

Suitable salts of carbonic acid include carbonic acid salts of one or more alkali metal, alkaline earth metal, ammonium and transition metal (for example manganese, iron, nickel, copper, zinc and silver). For example, suitable salts of carbonic acid include one or more of lithium carbonate, sodium carbonate, sodium bicarbonate, sodium sequicarbonate, potassium carbonate, potassium bicarbonate, magnesium carbonate, magnesium bicarbonate, ammonium carbonate and ammonium bicarbonate. More suitably, the salt of carbonic acid is selected from one or more of sodium carbonate, sodium bicarbonate, potassium carbonate, potassium bicarbonate, ammonium carbonate, ammonium bicarbonate. More suitably, the salt of carbonic acid is selected from one or more of sodium carbonate, sodium bicarbonate, potassium carbonate and potassium bicarbonate.

In some embodiments only a single salt of carbonic acid may be present in the solid cleansing composition of the first aspect of the invention. In some embodiments, a mixture of two or more salts of carbonic acid may be present. In such embodiments, the above amounts refer to the total amounts of all salts of carbonic acid present in the composition.

Suitably, the solid cleansing composition of the first aspect comprises from 20 to 40 wt% of the organic acid, more suitably from 22.5 to 37.5 wt% of the organic acid, such as from 25 to 35 wt% of the organic acid.

Suitably, the organic acid suitably comprises a monocarboxylic acid, a polycarboxylic acid, or a mixture thereof. The monocarboxylic acid or polycarboxylic acid suitably comprises from 2 to 8, such as from 4 to 6, carbon atoms. The monocarboxylic acid or polycarboxylic acid may comprise one or more hydroxy groups.

Suitably, the organic acid is selected from one or more of lactic acid, succinic acid, fumaric acid, salicylic acid, glycolic acid, ascorbic acid, maleic acid, malic acid, isocitric acid, and citric acid. More suitably, the organic acid is selected from one or more of isocitric acid, and citric acid. Stereoisomers of lactic acid, ascorbic acid, malic acid, and isocitric acid are also suitable.

In some embodiments only a single organic acid may be present in the solid cleansing composition of the first aspect of the invention. In some embodiments, a mixture of two or more organic acids may be present. In such embodiments, the above amounts refer to the total amounts of all organic acids present in the composition.

Suitably, the solid cleansing composition of the first aspect comprises: (i) from 5 to 50 wt% of the at least one acyl alkyl isethionate surfactant of the formula (I), (ii) from 30 to 60 wt% of the salt of carbonic acid, and (iii) from 20 to 40 wt% of the organic acid. More suitably, the composition comprises: from 10 to 45 wt% of the at least one acyl alkyl isethionate surfactant of the formula (I), (ii) from 35 to 57 wt% of the salt of carbonic acid, and (iii) from 22.5 to 37.5 wt% of the organic acid; such as from 10 to 35 wt% of the at least one acyl alkyl isethionate surfactant of the formula (I), (ii) from 40 to 55 wt% of the salt of carbonic acid, and (iii) from 25 to 35 wt% of the organic acid.

The solid cleansing composition of the first aspect may comprise at least one additional ingredient. By additional ingredient, we mean a component of the composition other than the acyl alkyl isethionate surfactant of the formula (I), the salt of carbonic acid, and the organic acid. Suitably, the composition comprises from 0.001 to 25 wt% of the at least one additional ingredient, such as from 0.01 to 25 wt%, for example from 0.1 to 25 wt%, of the at least one additional ingredient. For example, the composition may comprise from 1 to 20 wt% of the at least one additional ingredient, such as from 1 to 15 wt% of the at least one additional ingredient.

In some embodiments the additional ingredient comprises one or more further surfactants in addition to the acyl alkyl isethionate surfactant of the formula (I). Suitably, the solid cleansing composition of the first aspect of the invention comprises from 0.01 to 25 wt%, such as from 1 to 10 wt%, of an additional surfactant.

The additional surfactants may be selected from one or more anionic surfactants, cationic surfactants, non-ionic surfactants and amphoteric surfactants.

Suitable anionic surfactants for use in compositions of the first aspect of the invention include salts of C12-C18 carboxylic acids, ethoxylated carboxylic acids, ester carboxylates and ethoxylated ester carboxylates and sarcosinates. Other suitable anionic surfactants include sulfates and sulfonates, for example alkyl sulfates, alkyl ether sulfates, alcohol sulfates, alcohol ether sulfates, α-olefin sulfonates, linear alkyl sulfonates; and phosphate esters.

Suitable anionic surfactants may be selected from salts of fatty acids; alkali metal salts of mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; alkyl glyceryl ether sulfonates; alpha-olefinsulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, non-acylated alkyl isethionates; fatty acid taurates; acyl taurates; amino acid surfactants such as glutamates and glycinates; products of condensation of fatty acids with oxy- and aminoalkanesulfonic acids; sulfated derivatives of fatty acids and polyglycols; alkyl and acyl sarcosinates; sulfoacetates; alkyl phosphates; alkyl phosphate esters; acyl lactates; alkanolamides of sulfated fatty acids and salts of lipoamino acids. Particularly exemplary salts of the above, where applicable, are the sodium, potassium, ammonium, magnesium and triethanolamine salts.

Suitable sulfoacetates include acyl sulfoacetates, particularly sodium acyl sulfoacetates.

Acyl isethionates for use in compositions of the first aspect of the invention may be of the formula (II):

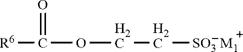

wherein R6 represents an optionally substituted C4-C36 hydrocarbyl group; and M1+ represents a cation.

Suitably, R6 represents an optionally substituted C4-C36 alkyl, C4-C36 alkenyl, C6-C12 aryl or C8-C22 alkyl-C6-C12 aryl group. More suitably, R6 represents an optionally substituted C4-C36 alkyl or C4-C36 alkenyl group. Most suitably, R6 represents a C4-C36 alkyl group or C4-C36 alkenyl group, especially a C4-C36 alkyl group.

Suitably, R6 represents an optionally substituted C5-C30 alkyl group, such as an optionally substituted C7-C24 alkyl group, for example an optionally substituted C7-C21 alkyl group, preferably an optionally substituted C7-C17 alkyl group.

Suitably, R6 represents a C5-C30 alkyl group, such as a C7-C24 alkyl group, for example a C7-C21 alkyl group, preferably a C7-C17 alkyl group.

R6 is suitably the residue of a fatty acid. Fatty acids obtained from natural oils often include mixtures of fatty acids. For example, the fatty acid obtained from coconut oil contains a mixture of fatty acids including C12 lauric acid, C14 myristic acid, C16 palmitic acid, C8 caprylic acid, and C18 stearic and oleic acid.

R6 may include the residue of one or more naturally occurring fatty acids and/or of one or more synthetic fatty acids. For example, R6 consists essentially of the residue of a single fatty acid.

Examples of carboxylic acids from which R6 may be derived include coco acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behinic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Suitably R6 comprises the residue of coco acid, the residue of mixed fatty acids derived from coconut oil or the residue of mixed fatty acids derived from palm kernel oil.

Suitably, M1+ represents a metal cation or an optionally substituted ammonium cation, preferably a metal cation. Suitable ammonium cations include NH4+ and the ammonium cation of triethanolamine. Suitable metal cations include alkali metal cations, for example sodium, lithium and potassium cations, and alkaline earth metal cations, for example calcium and magnesium cations. Preferably M1+ represents a zinc, potassium or sodium cation. Most preferably M1+ represents a sodium cation.

The skilled person will appreciate that when M1+ is a divalent metal cation two moles of anion will be present for each mole of cation.

In some embodiments only a single acyl isethionate of the formula (II) may be present in the solid cleansing composition of the first aspect. In some embodiments a mixture of two or more acyl isethionates of the formula (II) may be present.

For example, the acyl isethionates of the formula (II) may be selected from one or more of sodium lauroyl isethionate, sodium cocoyl isethionate and sodium myristoyl isethionate. Sodium cocoyl isethionate is especially preferred.

Preferred additional anionic detersive surfactants for use in compositions of the first aspect of the invention include alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Suitable non-ionic surfactants for use in compositions of the first aspect of the invention include alcohol alkoxylates such as alcohol ethoxylates, alcohol propoxylates, and ethylene oxide/propylene oxide copolymer derived surfactants, aliphatic esters, aromatic esters, sugar esters, especially sorbitan esters, alkyl polyglucosides, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides and amineoxides.

Suitable non-ionic surfactants may be selected from the following: reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl (C6-C22) phenol-ethylene oxide condensates, the condensation products of aliphatic (C8-C18) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as a polysaccharide amide; lactobionamides; and alkyl polysaccharide nonionic surfactants, for example alkylpolyglycosides.

Suitable cationic surfactants for use in compositions of the first aspect of the invention are typically based on fatty amine derivates or phosphonium quaternary ions, and quaternary ammonium compounds.

Suitable cationic surfactants for use in compositions of the first aspect of the invention include tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate.

Examples of suitable cationic surfactants include quaternary ammonium compounds, particularly trimethyl quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by halogen (e.g. bromide), acetate, citrate, lactate, glycolate, phosphate nitrate, sulfate, or alkylsulfate.

Further suitable cationic surfactants include those materials having the CTFA designations Quatemium-5, Quaternium-31 and Quaternium-18. Mixtures of any of the foregoing materials may also be suitable. A particularly useful cationic surfactant for use as a hair conditioning agent is cetyltrimethylammonium chloride, available commercially, for example as GENAMIN CTAC, ex Hoechst Celanese.

Salts of primary, secondary, and tertiary fatty amines are also suitable cationic surfactants. The alkyl groups of such amines preferably have from 12 to 22 carbon atoms, and can be optionally substituted.

Useful cationic surfactants include amido substituted tertiary fatty amines, in particular tertiary amines having one C12 to C22 alkyl or alkenyl chain. Such amines include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachid amidopropyldiethylamine, arachidamidoethyldiethylamine, arachidamidoethyldimethylamine, diethylaminoethylstearamide.

Also useful are dimethylstearamine, dimethylsoyamine, soyamine, myristylamine, tridecylamine, ethylstearylamine, Ntallowpropane diamine, ethoxylated (with 5 moles of ethylene oxide) stearylamine, dihydroxyethylstearylamine, and arachidyl behenylamine.

These amines are typically used in combination with an acid to provide the cationic species. Suitable acids include L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid.

Other useful cationic amine surfactants include those disclosed in US 4275055.

Suitable amphoteric surfactants for use in compositions of the first aspect of the invention include those based on fatty nitrogen derivates and those based on betaines.

Suitable amphoteric or zwitterionic surfactants may be selected from betaines, for example alkyl betaines, alkylamidopropyl betaines, for example cocamidopropyl betaine, alkylamidopropyl hydroxy sultaines, alkylamphoacetates, alkylamphodiacetates, alkyl propionates, alkylamphodipropionates, alkylamphopropionates, alkyliminodipropionates and alkyliminodiacetate.

Amphoteric or zwitterionic surfactants for use in compositions of the first aspect may include those which have an alkyl or alkenyl group of 7 to 22 carbon atoms and comply with an overall structural formula:

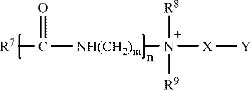

where R7 is alkyl or alkenyl of 7 to 22 carbon atoms; R8 and R9 are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO2 or —SO3.

Amphoteric or zwitterionic surfactants may include simple betaines of formula:

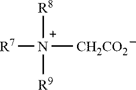

and amido betaines of formula:

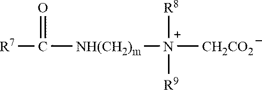

where m is 2 or 3.

In both formulae R7, R8 and R9 are as defined previously. R7 may, in particular, be a mixture of C12 and C14 alkyl groups derived from coconut so that at least half, preferably at least three quarters, of the groups R7 has 10 to 14 carbon atoms. R8 and R9 are preferably methyl.

Amphoteric or zwitterionic surfactants may include sulfobetaines of formula:

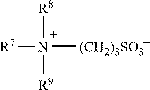

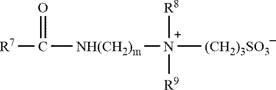

where m is 2 or 3, or variants of these in which —(CH2)3SO3— is replaced by

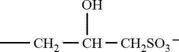

where R7, R8 and R9 in these formulae are as defined previously.

Amphoteric or zwitterionic surfactants may include amphoacetates and diamphoacetates. Amphoacetates generally conform to the following formula:

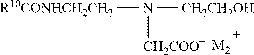

Diamphoacetates generally conform to the following formula:

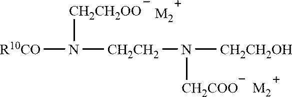

where R10 is an aliphatic group of 8 to 22 carbon atoms and M2+ is a cation such as sodium, potassium, ammonium, or substituted ammonium.

Suitable acetate-based surfactants include lauroamphoacetate; alkyl amphoacetate; sodium alkyl amphoacetate; cocoampho(di)acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate.

Suitable betaine surfactants include alkylamido betaine; alkyl betaine, C12/14 alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, C8 amido betaine; C12 amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C12/18 alkyl amido propyl dimethyl amine betaine; lauryldimethyl betaine; ricinol amidobetaine; tallow aminobetaine.

Suitable glycinate surfactants include acyl glycinates such as cocoamphocarboxyglycinate; tallowamphocarboxygycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C10/12 fatty acid amidoethyl-N-(2-hydroxyethyl)-glycinate; N—C12/18-fatty acid amidoethyl-N-(2-hydroxyethyl)-glycinate; dihydroxyethyl tallow gycinate.

Suitable glutamate surfactants include acyl glutamates.

The solid cleansing composition of the first aspect may comprise a chelating agent as an additional ingredient. Suitable chelating agents include ethylenediamine-N,N′-disuccinic acid, methylglycinediacetic acid, glutamic acid N,N-diacetic acid, imino disuccinic acid, diethylene triamine pentaacetic acid, ethylenediamine tetraacetic acid, diethylenetriamine penta methylene phosphonic acid, etidronic acid and anions and mixtures thereof.

Preferred chelants are biodegradable chelants for example ethylenediamine-N,N′-disuccinic acid, methylglycinediacetic acid, glutamic acid N,N-diacetic acid, imino disuccinic acid and anions and mixtures thereof.

Preferably, the solid cleansing composition of the first aspect comprises less than 10 wt% traditional soap compounds. By traditional soap compounds we mean to refer to compounds commonly known as soap, i.e. the alkali metal, alkaline earth metal, ammonium, ammonium hydroxide and alkanol ammonium salts of aliphatic alkane or alkene monocarboxylic acids.

Preferably the compositions of the first aspect comprise less than 5 wt% traditional soap compounds, preferably less than 2.5 wt%, more preferably less than 1 wt% traditional soap compounds. In some embodiments the compositions of the first aspect may be substantially free from traditional soap compounds.

By substantially free from traditional soap compounds we mean that such a product is not deliberately added to the composition. However, the skilled person will appreciate that fatty acids and salts thereof may be present in the composition as side products when providing other surfactants present in the composition, for example the compound of formula (I).

The composition of the first aspect may suitably comprise a conditioning agent as an additional ingredient. Suitable conditioning agents include cationic surfactants, cationic polymers and silicone conditioning agents. Suitable cationic surfactants are as previously defined herein. For example, the additional ingredient may comprise a cationic conditioning polymer.

Suitable cationic conditioning polymers include cationic polysaccharide polymers, copolymers of 1-vinyl-2-pyrrolidine and 1-vinyl-3-methylimidazolium salt (CTFA name Polyquaternium-16); copolymers of 1-vinyl-2-pyrrolidine and dimethylaminoethyl methacrylate, (CTFA name Polyquaternium-11); cationic diallyl quaternary ammonium-containing polymers in particular (CTFA Polyquaternium 6 and Polyquaternium 7), mineral acid salts of amino-alkyl esters of homo-and copolymers of unsaturated carboxylic acids, for example as described in US4009256; and cationic polyacrylamides, for example as described in W095/22311.

Cationic polysaccharide polymers suitable for use in compositions of the first aspect include those with an anhydroglucose residual group, such as a starch or cellulose. Cationic cellulose is available from Amerchol Corp. (Edison, NJ, USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, NJ, USA) under the tradename Polymer LM-200.

Other suitable cationic polysaccharide polymers include quaternary nitrogen-containing cellulose ethers (e. g. as described in U.S. Pat. 3,962, 418), and copolymers of etherified cellulose and starch (e. g. as described in U.S. Pat. 3,958, 581).

A particularly suitable type of cationic polysaccharide polymer that can be used is a cationic guar gum derivative, such as guar hydroxypropyltrimonium chloride or hydroxypropyl guar hydroxypropyltrimonium chloride (commercially available from Solvay in their JAGUAR trademark series). Particularly preferred cationic polymers are JAGUAR C13S, JAGUAR C14, JAGUAR C15, JAGUAR C17 and JAGUAR C16 Jaguar CHT, JAGUAR C162, Jaguar Excel and Jaguar C-500 STD.

The solid cleansing compositions of the first aspect may comprise a silicone conditioning agent as an additional ingredient.

Suitable silicone conditioning agents include polydiorganosiloxanes, in particular polydimethylsiloxanes that have the CTFA designation dimethicone. Also suitable for use in compositions of the first aspect (particularly shampoos and conditioners) are polydimethyl siloxanes having hydroxyl end groups, which have the CTFA designation dimethiconol. Also suitable for use in compositions of the first aspect are silicone gums having a slight degree of cross-linking, for example as described in WO 96/31188.

A further preferred class of silicones are amino functional silicones. By “amino functional silicone” is meant a silicone containing at least one primary, secondary or tertiary amine group, or a quaternary ammonium group.

Examples of suitable amino functional silicones include: polysiloxanes having the CTFA designation “amodimethicone”, Specific examples of amino functional silicones suitable for use in compositions of the first aspect are the aminosilicone oils DC2-8220, DC2-8166, DC2-8466, and DC2-8950- 114 (all ex Dow Corning), and GE 1149-75, (ex General Electric Silicones).

Suitable quaternary silicone polymers are described in EP530974. A preferred quaternary silicone polymer is K3474, ex Goldschmidt.

Also suitable are emulsions of amino functional silicone oils with non-ionic and/or cationic surfactant.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2- 8154 (all ex Dow Corning).

Also suitable are emulsions of amino functional silicone oils with non-ionic and/or cationic surfactant.

Pre-formed emulsions of amino functional silicone are also available from suppliers of silicone oils such as Dow Corning and General Electric. Specific examples include DC929 Cationic Emulsion, DC939 Cationic Emulsion, and the non-ionic emulsions DC2-7224, DC2-8467, DC2-8177 and DC2-8154 (all ex Dow Corning).

The solid cleansing composition of the first aspect of the invention may comprise a free fatty acid as an additional ingredient. These may be present in an amount of from 0.001 to 15 wt%, such as from 0.001 to 10 wt%, for example from 0.001 to 7 wt%, suitably from 0.001 to 5 wt%.

The solid cleansing composition of the present invention may include salts of fatty acids for example salts of monovalent and/or divalent metals.

Free fatty acids and salts of fatty acids may be provided as a side product of the compound of formula (I).

In some embodiments the at least one additional ingredient comprises a sodium acyl isethionate, a sodium alkyl amphoacetate, disodium cocoamphodiacetate, an alkyl betaine, an alkamidopropyl betaine, an alkamidopropyl hydroxyl sultaine, an alkyl propionate, an alkyl sulfate, an alkyl ether sulfate, an alkyl sulfosuccinate, an alkyl ether sulfosuccinate, an acyl taurate (such as sodium lauroyl methyl taurate), an acyl glycinate, an acyl glutamate, an acyl sarcosinate, an alkyl polyglucoside, an acyl lactylate, a sodium acyl sulfoacetate, an aliphatic ester, an aromatic ester, a glycerol ester, an alcohol alkoxylate, a fatty acid alkoxylate, a fatty acid, or a mixture thereof.

The at least one additional ingredient may include further optional ingredients for example fragrances, dyes, hair colourants such as semi-permanent dyes or pigments, hair growth agents, hair growth retardation agents, structuring aids, fillers, slipping agents, plasticising agents, anti-shrinkage agents, binding agents, flowing agents (to aid in processing before compressing into a bar), disintegrants (to aid the dissolution of particularly robust bars), moisturisers, sensory property agents such as cooling agents and warming agents, scalp exfoliant particles, beads or encapsulates which are physically robust in the solid form but rupture on contact with water, opacifying/pearlising agents (e.g. styrene/acrylates copolymer and ethylene glycol distearate), scalp benefit agents, colouring agents, sunscreens, UV filters, preservatives, penetration enhancers (e.g. propylene carbonate, benzyl alcohol etc.), hair styling agents which reside on the hair after rinsing to give the hair stylability and shape longevity, agents for the treatment and/or prevention of head and or pubic lice, agents for the eradication and/or repellence of ticks and other insect pests in human hair and/or animal hair/fur, fungicidal agents, bacteriocidal agents, yeasticidal agents, pH adjustment agents, foam boosting agents (such as cocamide DEA, cocamide MEA, or cocamide MIPA laureth-3), chelating agents, antidandruff agents, active ingredients (such as salicylic acid, benzoyl peroxide, sunscreen, botanical or antimicrobial agents), natural and vegetable oils (including hydrogenated and non-hydrogenated vegetable oils), electrolytes, waxes (such as paraffin, beeswax or Carnauba), polyquats (such as Polyquat 7, Polyquat 10, Polyquat 11 or Polyquat 22), and poloxamers. Components of this type are not limited to those mentioned and will be well known to the person skilled in the art.

The at least one additional ingredient may be selected from one or more of a vegetable oil, an electrolyte, a fragrance, a pigment/colourant, a filler, a wax, a polyquat, a poloxamer, a polyhydroxy alcohol, a chelant, and an active ingredient.

Suitable fillers include talc, starch, and maltodextrin.

Anti-dandruff agents include piroctone olamine, zinc pyrithione and salicylic acid.

Suitable electrolytes include ionic compounds, for example salts selected from sodium chloride, sodium sulfate, potassium chloride, potassium sulfate, sodium phosphate, disodium phosphate, potassium phosphate, dipotassium phosphate, sodium lactate, and sodium citrate. A preferred electrolyte is sodium chloride.

Suitable natural oils may act as an emollient and include coconut oil, sunflower oil, canola oil, hydrogenate canola oil, palm kernel oil, almond oil, apricot kernel oil, avocado oil and castor oil. Such natural oils may be included in the composition of the first aspect in an amount of 0.001 to 15 wt%.

The solid cleansing composition of the first aspect can be used to clean any suitable substrate, such as the skin or hair, for example the skin or hair of an animal or human. The solid cleansing composition of the first aspect may be provided as a single dose, for example for use in cleaning a desired substrate, such as the skin or hair, for example the skin or hair of an animal or human.

According to a second aspect of the present invention, there is provided a cleansing product comprising a solid cleansing composition according to the first aspect and packaging.

Any suitable packaging may be used and will depend on the exact nature of the product. Suitably, the packaging may be arranged so as to enable a single dose of the cleansing product to be packaged individually.

Suitably, the packaging is water-soluble. This allows the product of the second aspect to be applied by a user without removing the solid cleansing composition from its packaging. For example, the water-soluble packaging may comprise water-soluble cellulosic packaging or water-soluble starch packaging.

The product of the second aspect may include instructions for use. These may be provided on the packaging.

According to a third aspect of the present invention, there is provided a method of cleansing skin and/or hair comprising the steps of:
(a) contacting the composition according to the first aspect with water to form a cleansing foam; and
(b) contacting the cleansing foam with the skin and/or hair.

The method of the third aspect may suitably involve wetting the skin and/or hair and contacting the wet skin and/or hair with the composition according to the first aspect, thereby forming a cleansing foam in contact with the skin and/or hair. The method may suitably comprise forming a cleansing foam away from the skin and/or hair to be cleansed and subsequently contacting the cleansing foam with the skin and/or hair.

The method of the third aspect may include the step of rinsing the cleansing foam from the skin and/or hair with water.

Preferably, the method of the third aspect is a method of washing skin and/or hair.

According to a fourth aspect of the present invention, there is provided a use of a composition according to the first aspect for cleansing skin and/or hair.

According to a fifth aspect of the present invention, there is provided a method of manufacturing a solid cleansing product, the method comprising the steps of:
(a) forming an admixture comprising:
   (i) at least one acyl alkyl isethionate surfactant of the formula (I): 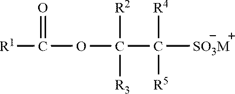
   wherein R1 represents an optionally substituted C4-C36 hydrocarbyl group; each of R2, R3, R4 and R5 independently represents hydrogen or a C1-C4 alkyl group and wherein at least one of R2, R3, R4 and R5 is not hydrogen; and M+ represents a cation;
   (ii) a salt of carbonic acid; and
   (iii) an organic acid; and
(b) enclosing the admixture in packaging.

The admixture may comprise at least one additional ingredient as defined in relation to the first aspect.

Step (a) of the method of the fifth aspect may comprise forming a mixture of at least one acyl alkyl isethionate surfactant of the formula (I), a salt of carbonic acid, an organic acid and optionally at least one additional ingredient using any suitable method and optionally compressing the mixture to provide a solid composition.

When step (a) comprises forming a mixture of at least one acyl alkyl isethionate surfactant of the formula (I) and optionally at least one additional ingredient, the powdered mixture so obtained may then be admixed with the salt of carbonic acid and the organic acid to form an admixture. Preferably the salt of carbonic acid and the organic acid are in a crystalline or powdered form, and the admixture is correspondingly in a crystalline or powdered form.

The crystalline or powdered admixture may be compressed to form a compacted admixture.

Step (b) comprises enclosing the admixture, which may be in a compacted, crystalline, or powdered form, in packaging. Preferred aspects of the packaging are as defined in relation to the second aspect.

Preferred features of the second, third, fourth and fifth aspects are as defined in relation to the first aspect.

### BRIEF DESCRIPTION OF DRAWINGS

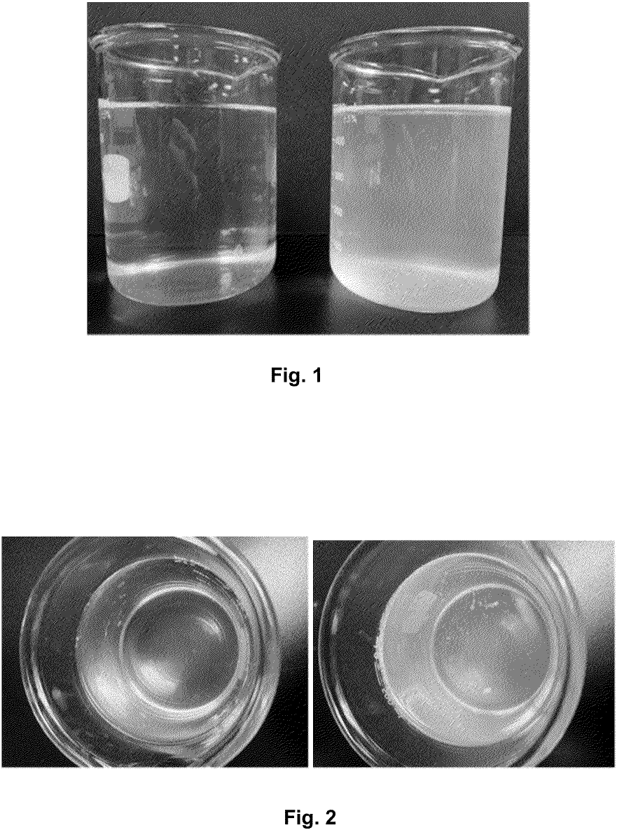

For a better understanding of the invention, and to show how exemplary embodiments of the same may be carried into effect, reference will be made, by way of example only, to the accompanying diagrammatic Figures, in which FIGS. 1 and 2 show the results of dissolution tests for the compositions prepared as Example 4 and Comparative A in the examples.

### EXAMPLES

The invention will now be described with reference to the following non-limiting examples.

Surfactant A - Sodium lauroyl methyl isethionate

This commercially available surfactant was provided as a powder containing 85 to 85 wt% of the active surfactant compound.

Surfactant B - Sodium lauroyl methyl taurate

This commercially available surfactant was provided as a powder containing 90 wt% of the active surfactant compound.

Surfactant C = Sodium lauryl sulfate

This commercially available surfactant was provided as a powder containing 98 wt% of the active compound.

Surfactant D = Sodium cocoyl isethionate

This commercially available surfactant was provided as a powder containing 78-80 wt% of the active surfactant compound.

### Example 1

A solid cleansing composition was prepared comprising the following ingredients, as shown in Table 1:

TABLE 1

| Component | % w/w |
|---|---|
| Citric Acid | 26 |
| Sodium Bicarbonate | 52 |
| Surfactant A | 12 |
| Surfactant B | 9 |
| Coconut Oil | 1 |

Procedure:
1. In the main mixing vessel, combine Citric Acid and Sodium Bicarbonate.
2. In a side vessel, mix Surfactant A, Surfactant B and Coconut oil until uniform and lump free.
3. Add side phase to the main vessel and mix until uniform.
4. Scoop one gram of the powdered mixture into pill press. Apply pressure to compress mixture into a pill/pellet

### Example 2

A solid cleansing composition was prepared comprising the following ingredients, as shown in Table 2:

TABLE 2

| Component | % w/w |
|---|---|
| Citric Acid | 26 |
| Sodium Bicarbonate | 52 |
| Surfactant A | 18 |
| Surfactant C | 3 |
| Coconut Oil | 1 |

Procedure:
1. In the main mixing vessel, combine Citric Acid and Sodium Bicarbonate.
2. In a side vessel, mix Surfactant A, Surfactant C and Coconut oil until uniform and lump free.
3. Add side phase to the main vessel and mix until uniform.
4. Scoop one gram of the powdered mixture into pill press. Apply pressure to compress mixture into a pill/pellet.

### Example 3

A solid cleansing composition was prepared comprising the following ingredients, as shown in Table 3:

TABLE 3

| Component | % w/w |
|---|---|
| Citric Acid | 26 |
| Sodium Bicarbonate | 52 |
| Surfactant A | 15 |
| Surfactant D | 6 |
| Coconut Oil | 1 |

Procedure:
1. In the main mixing vessel, combine Citric Acid and Sodium Bicarbonate .
2. In a side vessel, mix Surfactant A, Surfactant D and Coconut oil until uniform and lump free.
3. Add side phase to the main vessel and mix until uniform.
4. Scoop one gram of the powdered mixture into pill press. Apply pressure to compress mixture into a pill/pellet.

### Example 4

A solid cleansing composition was prepared (using the procedure set out above in relation to Example 3) comprising the following ingredients, as shown in Table 4:

TABLE 4

| Component | % w/w |
|---|---|
| Citric Acid | 26 |
| Sodium Bicarbonate | 52 |
| Surfactant A | 12 |
| Surfactant B | 9 |
| Coconut Oil | 1 |

A comparative composition (hereinafter “Comparative A”) was also prepared (using the procedure set out above in relation to Example 3) comprising the following ingredients, as shown in Table 5:

TABLE 5

| Component | % w/w |
|---|---|
| Citric Acid | 25.85 |
| Sodium Bicarbonate | 51.85 |
| Surfactant D | 12.31 |
| Surfactant B | 9 |
| Coconut Oil | 1 |

The compositions of Example 4 and Comparative A were tested for foaming ability.

Foaming ability was tested by a procedure in which a 0.5 w/w% of an aqueous solution was prepared

by dissolving 2.5 g of the composition (as a powder) in 500 ml of water. 100 ml of the solution

was added into a 500 cm3 measuring cylinder and shaken 10 times from head to waist. The height of

foam was then read. This was repeated so as to give 3 results for each composition tested and the

average foam height was calculated. The results are shown in Table 6.

TABLE 6

| | |
|---|---|
| Composition | Average foam height |
| Example 4 | 325 cm 10 |
| Comparative A | 275 cm |

The composition of Example 4 provided improved foaming compared to the composition of Comparative A.

The compositions of Example 4 and Comparative A were also subjected to a dissolution test.

The dissolution test was conducted by adding 1 g of each composition as a tablet to 500 g of water (to provide a 0.2% w/w solution). The formulations were allowed to dissolve without stirring (or any agitation) and were compared visually. The results are shown in FIGS. 1 and 2.

FIG. 1 shows side views of the formulation comprising the composition of Example 4 on the left and the formulation comprising the composition of Comparative A on the right. FIG. 2 shows vertical views of the formulation comprising the composition of Example 4 on the left and the formulation comprising the composition of Comparative A on the right. FIGS. 1 and 2 show that the composition of Example 4 dissolved in water to provide a clear solution at room temperature, whereas the composition of Comparative A provided a cloudy solution in water that included residue/particles at the bottom of the beaker.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A solid cleansing composition comprising:
(i) at least one acyl alkyl isethionate surfactant of the formula (I): 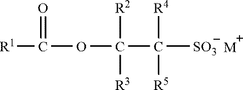
wherein R1 represents an optionally substituted C4-C36 hydrocarbyl group;
each of R2, R3, R4 and R5 independently represents hydrogen or a C1-C4 alkyl group and wherein at least one of R2, R3, R4 and R5 is not hydrogen; and M+ represents a cation;
(ii) a salt of carbonic acid; and
(iii) an organic acid.

2. A composition according to claim 1, which is effervescent.

3. A composition according to claim 1 or 2, comprising:
(i) from 5 to 50 wt% of the at least one acyl alkyl isethionate surfactant of the formula (I);
(ii) from 30 to 60 wt% of the salt of carbonic acid; and
(iii) from 20 to 40 wt% of the organic acid.

4. A composition according to any preceding claim, wherein R1 represents a C4-C36 alkyl or C4-C36 alkenyl group, preferably a C8-C18 alkyl or C8-C18 alkenyl group, and mixtures thereof.

5. A composition according to any preceding claim, wherein R2 represents a methyl group and R3, R4 and R5 each represent hydrogen, or R4 represents a methyl group and R2, R3 and R5 each represent hydrogen.

6. A composition according to any of claims 1 to 4, wherein the compound of formula (I) comprises a mixture of isomers including a compound of formula R1COOCH2CHR4SO3-M+ in which R4 represents a C1-C4 alkyl group and a compound of formula R1COOCHR2CH2SO3-M+ in which R2 represents a C1-C4 alkyl group.

7. A composition according to any preceding claim, wherein M+ represents a metal cation or an optionally substituted ammonium cation.

8. A composition according to any preceding claim, wherein the at least one acyl alkyl isethionate surfactant of the formula (I) is selected from one or more of sodium lauroyl methyl isethionate, sodium cocoyl methyl isethionate, sodium oleoyl methyl isethionate, and isomers thereof.

9. A composition according to any preceding claim, wherein the salt of carbonic acid is selected from one or more of sodium carbonate, sodium bicarbonate, potassium carbonate, potassium bicarbonate, ammonium carbonate, and ammonium bicarbonate.

10. A composition according to any preceding claim, wherein the organic acid is selected from one or more of a monocarboxylic acid and a polycarboxylic acid, suitably wherein the organic acid is selected from one or more of lactic acid, succinic acid, fumaric acid, salicylic acid, glycolic acid, ascorbic acid, maleic acid, malic acid, isocitric acid, and citric acid.

11. A composition according to any preceding claim, comprising at least one additional ingredient.

12. A composition according to claim 11, comprising from 0.001 to 25 wt%, preferably from 1 to 15 wt%, of the at least one additional ingredient.

13. A composition according to claim 11 or 12, wherein the at least one additional ingredient is selected from one or more of a cationic surfactant, a non-ionic surfactant, an anionic surfactant, and an amphoteric surfactant.

14. A composition according to any of claims 11 to 13, wherein the at least one additional ingredient is selected from one or more of a vegetable oil, an electrolyte, a fragrance, a pigment/colourant, a filler, a wax, a polyquat, a poloxamer, a polyhydroxy alcohol, a chelant, and an active ingredient.

15. A composition according to any preceding claim, which is substantially anhydrous.

16. A composition according to any preceding claim, which is in a compacted, crystalline or powdered form.

17. A cleansing product comprising a composition according to any preceding claim and packaging.

18. A cleansing product according to claim 17, wherein the packaging is water-soluble.

19. A cleansing product according to claim 17 or 18, wherein the packaging comprises a plurality of separate compartments.

20. A method of cleansing skin and/or hair comprising the steps of:
(a) contacting the composition according to any of claims 1 to 16 with water to form a cleansing foam; and
(b) contacting the cleansing foam with the skin and/or hair.

21. Use of a composition according to any of claims 1 to 16 for cleansing skin and/or hair.

22. A method of manufacturing a solid cleansing product, the method comprising the steps of:
(a) forming an admixture comprising:
(i) at least one acyl alkyl isethionate surfactant of the formula (I): 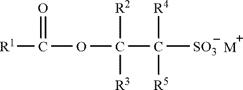
wherein R1 represents an optionally substituted C4-C36 hydrocarbyl group;
each of R2, R3, R4 and R5 independently represents a hydrogen atom or a C1-C4 alkyl group and wherein at least one of R2, R3, R4 and R5 is not hydrogen; and M+ represents a cation;
(ii) a salt of carbonic acid; and
(iii) an organic acid; and
(b) enclosing the admixture in packaging.
